(19) United States
(12) Patent Application Publication
(10) Pub. No.: US 2024/0009419 A1
(43) Pub. Date: Jan. 11, 2024

(54) GAS SUPPLY APPARATUS AND GAS SUPPLY METHOD

(71) Applicant: MiZ Company Limited, Kamakura-shi (JP)

(72) Inventors: Fumitake SATOH, Kamakura-shi (JP); Bunpei SATO, Kamakura-shi (JP); Yusuke ICHIKAWA, Kamakura-shi (JP)

(73) Assignee: MiZ Company Limited, Kamakura-shi (JP)

(21) Appl. No.: 18/449,144

(22) Filed: Aug. 14, 2023

Related U.S. Application Data
(63) Continuation of application No. PCT/JP2021/012307, filed on Mar. 24, 2021.

(51) Int. Cl. A61M 16/12; A61M 16/00; A61M 16/16; A61K 33/00; C25B 1/04; C25B 15/08; C25B 15/023

(52) U.S. Cl. CPC A61M 16/122; A61M 16/022; A61M 16/161; A61K 33/00; C25B 1/04; C25B 15/08; C25B 15/023; A61M 2202/0208; A61M 2205/3368

(57) ABSTRACT

There are provided a gas supply apparatus and a gas supply method that are capable of supplying hydrogen gas of a highly accurate and stable hydrogen gas concentration to a body. A ventilator 100 that supplies oxygen-containing gas and hydrogen gas includes a hydrogen gas delivery unit 8 that delivers the hydrogen gas taken in to outside of the ventilator 100, and a control unit 22 that adjusts an amount of the hydrogen gas taken into the hydrogen gas delivery unit 8. The control unit 22 adjusts the amount of the hydrogen gas taken into the hydrogen gas delivery unit 8 based on a flow rate of the oxygen-containing gas.

### RELATED APPLICATIONS

The present patent document is a continuation application of PCT Application Serial No. PCT/JP2021/012307, filed Mar. 24, 2021, designating the United States, which is hereby incorporated by reference.

### BACKGROUND

### Technical Field

The technology disclosed herein relates to a gas supply apparatus and a gas supply method.

### Background Art

The effectiveness of taking in hydrogen gas into the body is well known. As equipment for taking in hydrogen gas into the body, for example, Patent Document 1 discloses an in-body inhalation device for mixing hydrogen gas with air (oxygen-containing gas) for inhalation into the body. In this in-body inhalation device, the concentration of hydrogen gas to be inhaled in the body can be adjusted by mixing it with air, so that appropriate hydrogen gas can be inhaled according to the purpose.

### PATENT LITERATURE

[Patent Document 1]
Patent Publication No. 2009-005881

### BRIEF SUMMARY

### Problem to be Solved by the Invention

However, the in-body inhalation device disclosed in Patent Document 1 above does not control the amount of hydrogen gas generated in accordance with the flow rate of oxygen-containing gas, and thus cannot supply hydrogen gas of a highly accurate and stable hydrogen gas concentration.

The present invention was created in view of the above-mentioned points, and has as its object to provide a gas supply apparatus and a gas supply method capable of supplying hydrogen gas of a highly accurate and stable hydrogen gas concentration to the body.

### Means for Solving the Problem

The gas supply apparatus of the present invention is a gas supply apparatus that supplies oxygen-containing gas and hydrogen gas, and is provided with a hydrogen gas delivery unit that delivers said hydrogen gas taken in to the outside of said gas supply apparatus, and a control unit that adjusts the amount of said hydrogen gas taken into said hydrogen gas delivery unit. The control unit adjusts the amount of hydrogen gas taken into the hydrogen gas delivery unit based on the flow rate of the oxygen-containing gas.

The gas supply method of the present invention is a gas supply method for supplying oxygen-containing gas and hydrogen gas, comprising: a hydrogen gas generating process for generating said hydrogen gas by electrolysis of electrolyzed water; a mixing gas generating process for generating a mixed gas by mixing said hydrogen gas generated and taken in by said hydrogen gas generating process with said oxygen-containing gas. In the hydrogen gas generation process, the amount of hydrogen gas to be generated is adjusted by controlling the value of the electric current flowing in the electrolysis based on the flow rate of the oxygen-containing gas to be mixed with the hydrogen gas.

### BRIEF DESCRIPTION OF THE DRAWINGS

FIG. 1 A block diagram showing a configuration of a ventilator in accordance with a first embodiment.

FIG. 2 A cross-sectional view showing a configuration of a hydrogen gas delivery unit in the first embodiment.

FIG. 3(A) is a graph showing the relationship between the flow rate of oxygen-containing gas and time in the first embodiment. FIG. 3(B) is a graph showing the relationship between the current value and time in the first embodiment.

FIG. 4 A flowchart showing the process performed by the control unit in the first embodiment.

FIG. 5 A block diagram showing the configuration of a ventilator in a second embodiment.

FIG. 6 A cross-sectional view showing the configuration of the hydrogen gas delivery unit in accordance with the second embodiment.

FIG. 7 A block diagram showing the configuration of a ventilator according to a third embodiment.

FIG. 8 A cross-sectional view showing the configuration of the hydrogen gas delivery unit in accordance with the third embodiment.

FIG. 9 A block diagram showing the configuration of a ventilator according to a fourth embodiment.

FIG. 10 A block diagram showing the configuration of a ventilator according to a fifth embodiment.

FIG. 11 A block diagram showing the configuration of a ventilator in accordance with a sixth embodiment.

FIG. 12 A schematic diagram of an electrolyzer according to another embodiment.

### DETAILED DESCRIPTION OF THE DRAWINGS AND THE PRESENTLY PREFERRED EMBODIMENTS

### Description of Embodiments

### First Embodiment

With reference to the following drawings, a ventilator (an example of a gas supply apparatus) 100 according to the first embodiment will be described in detail. The ventilator 100 is a device that supplies oxygen-containing gas and hydrogen gas to a living body, and supplies inhaled air to be inhaled into the living body, that is, a mixed gas of oxygen-containing gas and hydrogen gas, to the living body, and also takes exhaled air from the living body and discharges it to the outside. Hereinafter, the mixed gas of oxygen-containing gas and hydrogen gas is simply referred to as “mixed gas” unless otherwise specified.

In this specification, “living body” refers to the living body of mammals, including dogs, cats, etc., in addition to humans, and the present invention is a device that can be used in the treatment of mammals in veterinary medicine as well as in the treatment of humans in medicine and dentistry.

The expression “hydrogen gas taken into the hydrogen gas delivery unit” referred to herein is not limited to hydrogen gas generated in the hydrogen gas delivery unit, but may, for example, include hydrogen gas delivered from outside the hydrogen gas delivery unit into the hydrogen gas delivery unit.

In this specification, the manner in which the mixed gas delivered from the ventilator 100 is inhaled into a living body is not limited to inhalation of the mixed gas through the nasal cavity or oral cavity via a mask or the like, but may also include, for example, direct delivery of the mixed gas into the trachea through a tracheotomy.

In this specification, “hydrogen,” the active ingredient of the compositions, is molecular hydrogen (i.e., gaseous hydrogen) and is referred to simply as “hydrogen” or “hydrogen gas” unless otherwise specified. The term “hydrogen” as used herein also refers to H₂, D₂ (deuterium), HD (deuterated hydrogen), or mixtures of these gases in molecular form; D₂ is more expensive, but is known to be a stronger superoxide scavenger than H₂. Hydrogen that can be used herein is H₂, D₂ (deuterium), HD (deuterated hydrogen), or a mixture of these gases. Preferably, H₂, or D₂ and/or HD may be used instead of or mixed with H₂.

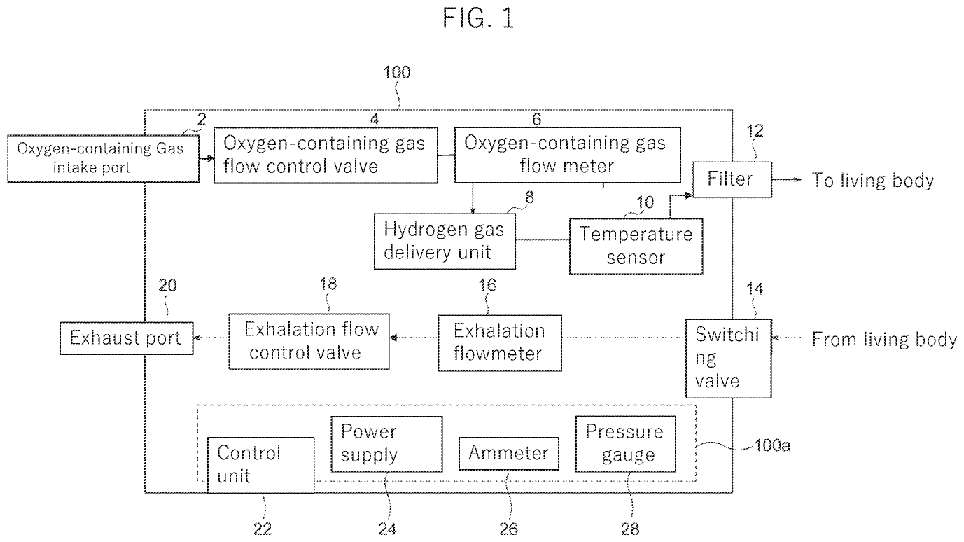

First, the overall configuration of the ventilator 100 of this embodiment will be described. As shown in FIG. 1, the ventilator 100 includes an inhalation path mechanism (a mechanism provided along the arrows shown in solid lines) that supplies a mixed gas to the living body, an exhalation path mechanism (a mechanism provided along the arrows shown in broken lines) that takes in and discharges exhaled air from the living body, and a control system mechanism 100a. The exhalation pathway mechanism includes an oxygen-containing gas intake port 2, an oxygen-containing gas flow control valve 4, an oxygen-containing gas flow meter 6, a hydrogen gas delivery unit 8, a temperature sensor 10, and a filter 12. The exhalation pathway mechanism is equipped with a switching valve 14, an exhalation flow meter 16, an exhalation flow control valve 18, and an exhaust port 20.

The oxygen-containing gas intake port 2 includes a port (not shown) for taking in compressed oxygen through a filter (not shown) from a cylinder or the like filled with compressed oxygen and a port (not shown) for taking in compressed air through a filter (not shown) from a cylinder or the like filled with compressed air. The compressed air may be compressed air taken in directly from outside. Compressed oxygen and compressed air taken in from both ports are mixed and delivered as oxygen-containing gas to the oxygen-containing gas flow control valve 4. The oxygen-containing gas taken in from the oxygen-containing gas intake port 2 is delivered to the hydrogen gas delivery unit 8 via the oxygen-containing gas flow control valve 4 and oxygen-containing gas flow meter 6. The cylinders and other equipment described above may be connected to the ventilator 100 from outside the ventilator 100 or may be integrated with the ventilator 100.

The oxygen-containing gas flow rate control valve 4 is a valve that adjusts the flow rate of oxygen-containing gas in order to deliver a predetermined amount of oxygen-containing gas to the hydrogen gas delivery unit 8 within a predetermined time. The oxygen-containing gas flow control valve 4 has a valve that opens and closes, for example, under the control of the control unit 22 described below, to adjust the flow rate of the oxygen-containing gas. The oxygen-containing gas flow meter 6 detects the flow rate of the oxygen-containing gas delivered from the oxygen-containing gas flow control valve 4 to the hydrogen gas delivery unit 8, converts the detected flow rate into an electrical signal, and transmits it to the control unit 22.

The hydrogen gas delivery unit 8 is a unit that takes in and delivers hydrogen gas generated by electrolysis of electrolyzed water, for example. The hydrogen gas taken in by the hydrogen gas delivery unit 8 is mixed with oxygen-containing gas and delivered as a mixed gas to the filter 12. Details of the hydrogen gas delivery unit 8 are described below. Temperature sensor 10 detects the temperature of the mixed gas delivered from hydrogen gas delivery unit 8 to filter 12, converts the detected temperature into an electrical signal, and transmits the signal to a control unit 22 described below.

Filter 12 is provided at the outlet from which the mixed gas is delivered from respirator 100 to remove dust and bacteria. The mixed gas that passes through the filter 12 is taken into the living body via a mask or other means.

The switching valve 14 is a valve that switches the direction of gas flow to allow only inhalation from the ventilator 100 to the living body and only exhalation from the living body to the ventilator 100. The switching valve 14 has a valve that opens and closes, for example, under the control of the control unit 22 described below, and the direction of gas flow is controlled by the opening and closing of this valve. The expiratory flow meter 16 detects the flow rate of expiratory air delivered from the switching valve 14 to the expiratory flow control valve 18, converts it into an electrical signal, and transmits it to the control unit 22.

The expiratory flow control valve 18 is a valve for adjusting the pressure of expiratory air discharged from the body. The expiratory flow control valve 18 has a valve that opens and closes, for example, under the control of the control unit 22 described below, to adjust the pressure of the gas discharged outside the ventilator 100. Gas passing through the expiratory flow control valve 18 is exhausted to the outside through the exhaust port 20.

The control system mechanism 100a has a control unit 22, a power supply 24, an ammeter 26, and a pressure gauge 28. The control unit 22 is a processing unit that controls the entire element comprising the ventilator 100 and is composed of a processor, a memory device, and the like. The control unit 22 executes the process described below based on a program stored in the memory device. The power supply 24 applies a voltage to the electrolyzer 30, which will be described later, thereby passing an electric current through the electrolyzer 30. The ammeter 26 detects the current value flowing in the electrolyzer 30 from the voltage applied to the electrolyzer 30. The pressure gauge 28 detects the airway pressure of the user using the ventilator 100.

An example of the process performed by the control unit 22 is described in detail. The control unit 22 controls the oxygen-containing gas flow control valve 4 so that the oxygen-containing gas is inhaled at a flow rate predetermined for each user. For example, a predetermined flow rate (liters/s) or flow pattern of oxygen-containing gas for each user is stored in the control unit 22, and the control unit 22 controls the oxygen-containing gas flow control valve 4 according to the stored flow rate (flow pattern).

The control unit 22 controls the switching valve 14 according to a predetermined cycle of inhalation and exhalation times. For example, the inhalation and exhalation times predetermined for each user are stored in the control unit 22, and the control unit 22 controls the switching valve 14 according to the stored times.

The control unit 22 controls the expiratory flow control valve 18 to discharge gas at a predetermined flow rate for each user. For example, a predetermined pressure for each user is stored in the control unit 22, and the control unit 22 controls the expiratory flow control valve 18 according to the stored pressure.

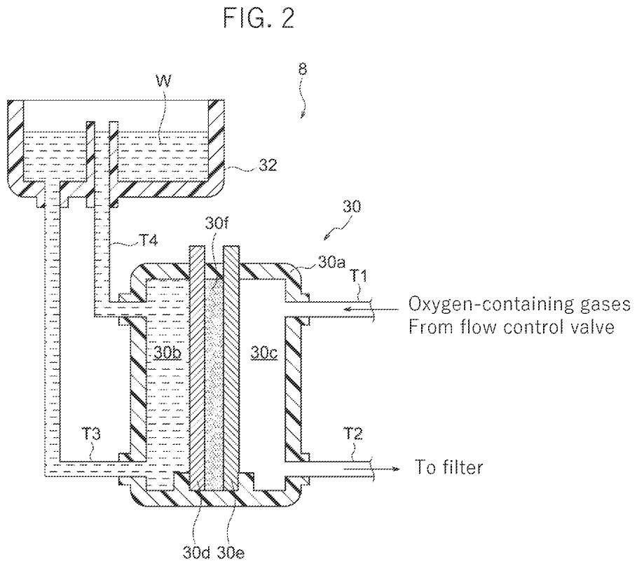

Referring now to FIG. 2, the configuration of the hydrogen gas delivery unit 8 is described in detail. As shown in FIG. 2, the hydrogen gas delivery unit 8 is composed of an electrolyzer 30 and a water tank 32. The electrolyzer 30 is a device for generating hydrogen gas by applying voltage between a pair of electrode plates (an example of a pair of electrodes) 30d, 30e provided inside and electrolyzing the electrolyzed water W flowing in the electrolyzer 30. The electrolyzer 30 has a housing 30a, a first chamber 30b, a second chamber 30c, a pair of electrode plates 30d (anodes), 30e (cathodes), and a diaphragm 30f. The housing 30a is formed of an electrically insulating material such as plastic, and is kept watertight or airtight except for the inlet/outlet of the electrolyzed water W and gas as described below.

The interior of the housing 30a is separated into a first chamber 30b and a second chamber 30c by the diaphragm 30f. The first chamber 30b has an electrode plate 30d inside that is in contact with the diaphragm 30f, and is a space into which electrolyzed water W is introduced and filled to generate hydrogen gas. The anode (+pole) of the power supply 24 is connected to the electrode plate 30d in the first chamber 30b. An ammeter 26 is provided between the power supply 24 and the electrode plate 30d, and the ammeter 26 can detect the electrical quantity of the current flowing through the electrode plate 30d.

The second chamber 30c has an electrode plate 30e inside that is in contact with the diaphragm 30f, and is a space where hydrogen gas generated from the electrolyzed water W is taken in. The cathode (−pole) of the power supply 24 is connected to the electrode plate 30e in the second chamber 30c. The oxygen-containing gas delivered from the oxygen-containing gas flow control valve 4 flows into the second chamber 30c through the intake pipe T1. In this embodiment, the hydrogen gas taken into the second chamber 30c and the oxygen-containing gas that flows into the second chamber 30c are mixed in the second chamber 30c and delivered to the filter 12 through an air supply pipe T2 as a mixed gas. The air supply pipe T2 is equipped with a gas outflow prevention mechanism (e.g., valve or cap), which is not shown in the figure, and when the gas outflow prevention mechanism is closed, the outflow of gas from the second chamber 30c is prevented.

The pair of electrode plates 30d and 30e, for example, may be made of titanium plates and coated with one or three or more precious metals selected from the group including platinum, iridium, and palladium. However, it is not limited only to these materials; for example, a solid stainless steel plate may be used.

The diaphragm 30f should be a cation exchange membrane that allows hydrogen ions to permeate, but not hydroxyl ions. In particular, an all-fluorinated sulfonic acid membrane with a sulfonic acid group as an electrophilic group is suitable, considering various factors such as ion conductivity, physical strength, gas barrier properties, chemical stability, electrochemical stability, and thermal stability. Such membranes include Nafion membrane (registered trademark, Du Pont), a copolymer membrane of perfluorovinyl ether and tetrafluoroethylene with sulfonic acid groups, Flemion membrane (registered trademark, Asahi Glass), Aciplex membrane (registered trademark, Asahi Glass) and others.

When the first chamber 30b is filled with electrolyzed water W, voltage is applied between a pair of electrode plates 30d and 30e from the power supply 24, and current flows between the pair of electrode plates 30d and 30e, the reaction of the following formula (1) occurs at the electrode plate 30d (anode) and the reaction of the following formula (2) occurs at the electrode plate 30e (cathode).

2OH⁻→H₂O+O₂/2+2e⁻ Formula (1)

2H₂O+2e⁻→H₂+2OH⁻ Formula (2)

As a result, oxygen gas (O₂) is produced from the electrode plate and hydrogen gas (H₂) is produced from the electrode plate 30e.

The water tank 32 is provided above the electrolyzer 30, and the electrolyzed water W is stored inside. The water tank 32 is connected to the lower part of the first chamber 30b of the electrolyzer 30 through the water supply pipe T3 and to the upper part of the first chamber 30b of the electrolyzer 30 through the exhaust pipe T4. Electrolyzed water W in the water tank 32 flows into the first chamber 30b of the electrolyzer 30 through the water supply pipe T3. The oxygen gas generated by the electrode plate 30d in the first chamber 30b is exhausted from the water tank 32 to the outside through the exhaust pipe T4.

Electrolyzed water W may include tap water, clean water, purified water, ion-exchanged water, RO water, distilled water, etc. The electrolyzed water W may contain electrolytes such as sodium ions, potassium ions, calcium ions, magnesium ions, etc. as appropriate. However, in order to avoid the generation of excess gases other than hydrogen and oxygen gases during electrolysis, it is desirable to artificially add water-soluble compounds to pure water containing no ions other than hydrogen and hydroxyl ions, such as ion-exchanged water and purified water, to make the water to be electrolyzed. In particular, chlorine gas is basically considered to be not beneficial to living organisms, so the electrolyzed water W stored in the water tank 32 in this embodiment should be treated to remove chlorine ions.

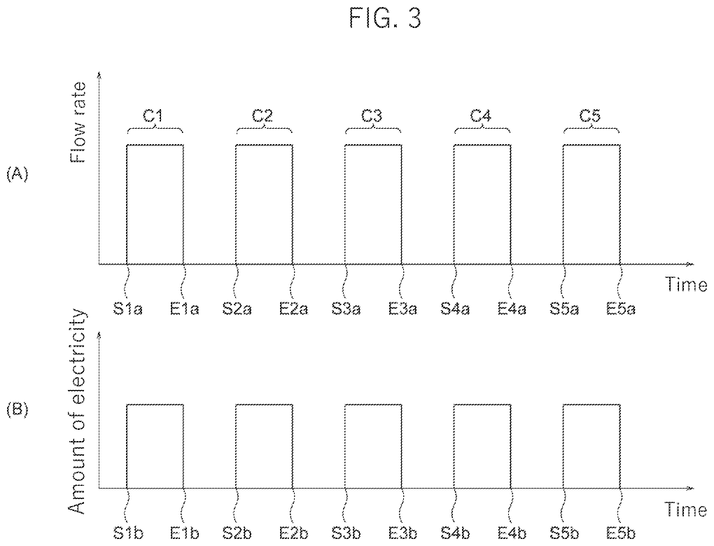

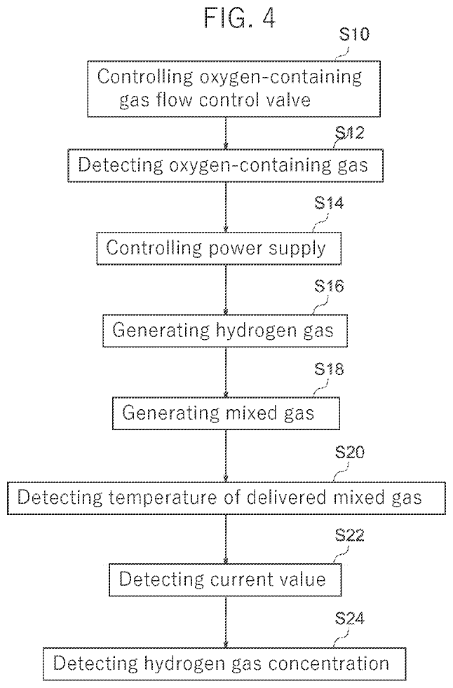

Referring to FIGS. 3(A), 3(B), and 4, the current value control process performed by the control unit 22 in this embodiment is explained next. The time axes in FIGS. 3(A) and 3(B) coincide. In the ventilator 100 of this embodiment, inhalation and exhalation are repeated in a predetermined cycle, and in one ventilation (one cycle of inhalation and exhalation), a predetermined amount of oxygen-containing gas is inhaled into the living body within a predetermined time. As shown in FIG. 3(A), in the first inhalation cycle C1, inhalation begins at the flow start point Si a, and oxygen-containing gas is inhaled into the living body at a constant flow rate. When the amount of inhaled oxygen-containing gas reaches a certain level, the flow rate of the oxygen-containing gas becomes zero at the flow rate end point E1a, and inhalation is terminated. After a predetermined time elapses after the first inhalation cycle C1, the second inhalation cycle C2 begins. The same flow rate change process as in C1 is executed in the second and subsequent inhalation cycles C2 to C5.

In this embodiment, the control unit 22 controls the current value flowing from the power supply 24 to the pair of electrode plates 30d, 30e of the electrolyzer 30 based on the change in the flow rate of the oxygen-containing gas shown in FIG. 3(A). As shown in FIG. 3(B), the current value flowing in the electrolyzer 30 increases and decreases according to the change in the flow rate of the oxygen-containing gas on the same time axis as shown in the graph in FIG. 3(A). In other words, as soon as the first inhalation cycle C1 starts, a constant current flows through the pair of electrode plates 30d, 30e at the current start point 51b, and the current stops at the current end point El b as soon as inhalation cycle C1 is completed. In the second and subsequent inhalation cycles C2 to C5, the current value changes in a similar manner.

The flowchart shown in FIG. 4 illustrates the process performed by control unit 22 to control the current value and oxygen-containing gas flow rate in one inhalation cycle C1-C5 shown in FIGS. 3(A) and 3(B). When inhalation cycles C1-C5 are started, control unit 22 first transmits a control signal to the oxygen-containing gas flow control valve 4 to control the opening and closing of the oxygen-containing gas flow control valve 4 (step S10). Through this control, oxygen-containing gas flows into the electrolyzer 30 at a flow rate according to the waveform shown in FIG. 3(A), and is mixed with the hydrogen gas produced in step S16 and inhaled into the living body.

Next, the control unit 22 detects the flow rate of the oxygen-containing gas delivered from the oxygen-containing gas flow control valve 4 to the electrolyzer 30 by means of an electrical signal transmitted from the oxygen-containing gas flow meter 6 (step S12). In this process, the control unit 22 may detect the instantaneous flow rate of the oxygen-containing gas, or it may detect the average flow rate from the integrated flow rate that has flowed within a predetermined time period.

Next, the control unit 22 calculates the current value (A) required to generate hydrogen gas in an amount corresponding to the flow rate of the oxygen-containing gas based on the flow rate of the oxygen-containing gas detected in step S12. The power supply 24 is then controlled so that a current of the calculated current value flows to the pair of electrode plates 30d, 30e in the electrolyzer 30 (step S14).

The method of calculating the current value performed by the control unit 22 in step S14 is described here. In this embodiment, the control unit 22 determines the target production volume of hydrogen gas per unit time (liters/s) according to the detected flow rate of oxygen-containing gas, and calculates the current value from the determined target production volume of hydrogen gas.

As a method for determining the target production volume of hydrogen gas, for example, a table defining the correspondence between the flow rate of oxygen-containing gas and the target production volume of hydrogen gas is stored in the control unit 22 in advance, and the control unit 22 may determine the target production volume of hydrogen gas corresponding to the flow rate of oxygen-containing gas detected in step S12 by reading from the said table. Or, for example, a calculation formula showing the proportional relationship between the flow rate of the oxygen-containing gas and the target production volume of hydrogen gas is stored in the control unit 22, and the control unit 22 may calculate and determine the target production volume of hydrogen gas by substituting the flow rate of the oxygen-containing gas detected in step S12 into the aforementioned proportional formula. The control unit 22 then calculates the current value as follows.

According to Faraday's second law of electrolysis, the electric quantity required to deposit an equal amount of substance per gram is constant regardless of the type of substance, and the following equation (3) holds:

n=m/M=It/zF Equation (3)
where n (mol) is the amount of substance, m (g) is the mass, M (g/mol) is the molecular weight, I (A) is the electric current, t (s) is the time the current flows, z is the ion valence, and F is Faraday constant F (=9.65×10 4 (C/mol)).

Since the electric quantity Q(C)=current I(A)×time t(s), the following equation (4) can be obtained from the above equation (3).

Q=It=nzF Equation (4)

Therefore, since the amount of electron substances required to generate 1 mol of hydrogen gas H₂ is 2 mol from the above formula (2), the electric quantity Q(C) required to produce n=1 mol of hydrogen gas is obtained by substituting n=2, the ionic valence of hydrogen z=1, and Faraday constant F=9.65×10⁴ (C/mol) can be substituted to obtain:

Q=2×9.65×10⁴(C) Equation (5)

When hydrogen gas is an ideal gas, the volume occupied by 1 mol of hydrogen gas is 22.4 (liters) under the standard condition of 0° C. temperature and 1 atm pressure, so the following relationship between the target production volume of hydrogen gas V₁ (liters/s) and the current value I₁ (A) required to produce the target production volume of hydrogen gas V₁ is established.

22.4/t: V₁=2×9.65×10⁴/t: I₁

By rearranging this relational equation, the following equation (6) can be obtained.

I₁=V₁×2×9.65×10⁴/22.4 (A) Equation (6)

In step S14, the control unit 22 calculates the required current value I₁ by applying the target production volume of hydrogen gas to the above equation (6), and controls the power supply 24 according to the current value I₁.

The process of step S14 is executed and an electric current flows to the electrode plates 30d and 30e of the electrolyzer 30, causing an electrolysis reaction in the electrolyzer 30 and generating hydrogen gas in an amount corresponding to the flow rate of the oxygen-containing gas detected in step S12 (step S16). The generated hydrogen gas is mixed in the second chamber 30c with the oxygen-containing gas that flows into the second chamber 30c of the electrolyzer 30 to generate a mixed gas (step S18). The process performed by the control unit 22 in steps S14 and S16 is an example of the hydrogen gas generation process, and the process performed by the control unit 22 in step S18 is an example of the mixed gas generation process.

Next, the control unit 22 detects the temperature of the mixed gas delivered from the electrolyzer 30 to the filter 12 by the electrical signal transmitted from the temperature sensor 10 (step S20). Next, the control unit 22 detects the current value (electrical quantity per unit time) of the current flowing to the electrode plate 30e (cathode) of the electrolyzer 30 by the electrical signal transmitted from the ammeter 26 (step S22). In this process, the control unit 22 may detect the instantaneous current value of the current flowing to the electrode plate 30e, or it may detect the electric quantity per unit time (=current value) from the integrated electric quantity within a predetermined time.

Next, the control unit 22 calculates the concentration of hydrogen gas in the mixed gas delivered from the electrolyzer 30 to the filter 12 (step S24). Specifically, the control unit 22 calculates the volume concentration of hydrogen gas (vol %) based on the flow rate of the oxygen-containing gas (liters/s) detected in step S12, the temperature of the mixed gas (K) detected in step S20, and the current value (A) detected in step S22. The control unit 22 calculates the volume concentration of hydrogen gas as follows.

From the above equation (5), if V₂ (liters) is the volume of hydrogen gas produced by 1 (C) of electricity in the standard state, the following relationship is established.

22.4:V₂=2×9.65×10⁴:1

This relational equation can be rearranged to obtain V₂=1.16×10⁻⁴ (liters).

According to Charles' law, the volume (liters) of an ideal gas increases or decreases by 1/273 of the volume (liters) at the state of 0° C. (≈273K) for every 1 (K) increase or decrease in temperature. Therefore, if the differential temperature (T₁-0) when the temperature of hydrogen gas increases from the state of 0° C. (≈273K) to the state of T₁° C. is ΔT(K), the volume V₃ (liters) of hydrogen gas produced at T₁° C. with 1 (C) of electricity is obtained as follows.

V₃=V₂+V₂(ΔT×1/273)=V₂(1+ΔT/273) Equation (7)

Therefore, the volume V_{H} (liters) of hydrogen gas produced at T₁° C. with an electrical quantity of Q (C) can be obtained from the above equation (7) as in equation (8) below.

V_{H}=Q×V₂(1+ΔT/273)=It×1.16×10⁻⁴×(1+ΔT/273) Equation (8)

As described above, in the second chamber 30c of the electrolyzer 30, the hydrogen gas produced is mixed with the oxygen-containing gas flowing into the second chamber 30c through the intake pipe T1, which dilutes the concentration of the hydrogen gas. If the flow rate of the oxygen-containing gas flowing through intake pipe T1 is Fₒ (liters/s), since the volume Vₒ of the oxygen-containing gas flowing into the second chamber 30c in 1 second (unit time) is equal to the flow rate Fₒ, and the volume per unit time of the mixed gas produced by mixing hydrogen gas and oxygen-containing gas in the second chamber 30c is V_{H}+Vₒ=V_{H}+Fₒ, the following equation (9) is established.

Volume concentration of hydrogen gas (vol %)=(V_{H}/(V_{H}+F_{O}))×100 Equation (9)

The control unit 22 calculates V_{H} by substituting t=1 (s), the temperature ΔT (K) of the mixed gas detected in step S20, and the current value I (A) detected in step S22 into the above equation (8). Then, the volume concentration of hydrogen gas in the mixed gas delivered from the hydrogen gas delivery unit 8 is calculated by substituting the value of V_{H} and the flow rate Fₒ of the oxygen-containing gas detected in step S12 into the above equation (9).

The effect of the temperature ΔT of the mixed gas on the volume concentration of hydrogen gas is a change of 1/273 per 1° C. of temperature, which is very small. Therefore, instead of detecting the temperature of the mixed gas by the temperature sensor 10, the volume concentration of hydrogen gas may be calculated by applying a constant value of temperature to ΔT in the above equation (8).

In the ventilator 100 of this embodiment, the target production volume of hydrogen gas is set so that the volume concentration of hydrogen gas delivered from the hydrogen gas delivery unit 8 is greater than 0 vol % and equal to or less than the detonation limit of 18.3 vol %. Therefore, in this embodiment, the control unit 22 controls the current values flowing to the electrode plates 30d and 30e of the electrolyzer 30 so that the volume concentration of the hydrogen gas taken into the second chamber 30c of the electrolyzer 30 (hydrogen gas produced in the electrolyzer 30) is within the above range. In this case, considering the risk of detonation or explosion, the volume concentration of hydrogen gas preferably not exceed 18.3 vol %, more preferably not exceed 10 vol %. If the hydrogen gas concentration calculated in step S24 is within the predetermined range (e.g., within the above range), the control unit 22 moves to the next inhalation cycle.

In this embodiment, if the volume concentration of hydrogen gas calculated in step S24 exceeds a certain value, the control unit 22 may control the power supply 24 to reduce the current value flowing to the electrode plates 30e of the electrolyzer 30 or to stop applying voltage to the electrode plates 30e. If the concentration of hydrogen gas exceeds a certain value, the control unit 22 may control the gas outflow prevention valve in the air supply pipe T2 to prevent hydrogen gas from being delivered outside the electrolyzer 30 and to allow hydrogen gas to be discharged from the electrolyzer 30 through an exhaust pipe (not shown).

If the volume concentration of hydrogen gas calculated in step S24 is extremely low (almost 0° C.), the control unit 22 may control the power supply 24 to stop the application of voltage to the electrode plates 30d, 30e because the electrolyzer 30 may be faulty.

As described above, in the ventilator 100 of this embodiment, the control unit 22 adjusts the amount of hydrogen gas taken into the hydrogen gas delivery unit 8 by controlling the current value flowing to the pair of electrode plates 30d, 30e based on the flow rate of oxygen-containing gas. Thus, the amount of hydrogen gas produced is increased or decreased according to the increase or decrease in the flow rate of the oxygen-containing gas. In the ventilator 100, the control unit 22 calculates the concentration of hydrogen gas after it is taken in and controls the concentration so that it does not exceed a certain value. As a result, the ventilator 100 can supply hydrogen gas of a highly accurate and stable hydrogen gas concentration to the body.

In the ventilator 100 of this embodiment, hydrogen gas is generated by electrolyzing the electrolyzed water W, so the temperature and humidity of the generated hydrogen gas are maintained at a constant level. Therefore, the mixed gas can be supplied to the living body at a temperature of at least 35° C. to 40° C. and a relative humidity of 100% or more at each temperature. As a result, depending on the health condition of the user, it may be unnecessary to install an artificial nose with heating and humidification functions or a heating and humidifying device, which is conventionally required for ventilators.

Since the temperature of the mixed gas is detected by the temperature sensor 10 in the ventilator 100 of this embodiment, the control unit 22 may control the current value flowing to the pair of electrode plates 30d, 30e based on the detected temperature. By controlling the current value, the temperature of the mixed gas produced in the electrolyzer 30 can be adjusted.

When controlling the current value based on temperature in this manner, the temperature of at least one of the gases, oxygen-containing gas, hydrogen gas, or mixed gas, may be detected by changing the position of the temperature sensor 10, and the current value may be controlled based on the detected temperature.

In the electrolyzer 30, when electrolysis proceeds by passing a current through a pair of electrode plates 30d, 30e, the temperature of the hydrogen gas generated in the electrolyzer 30 rises because the diaphragm 30f and the electrode plates 30d, 30e generate heat due to resistance caused by the passage of current, and the temperature of the electrolyzer 30 rises (for example, if the current value is constant, the temperature of the hydrogen gas generated rises by 20° C. or more during the period from the start of electrolysis until 6 hours have passed). Therefore, by decreasing the current value flowing to the pair of electrode plates 30d, 30e, the amount of heat generated by the diaphragm 30f and the electrode plates 30d, 30e decreases, and the temperature rise of the hydrogen gas generated in the electrolyzer 30 can be suppressed.

Here, the temperature of the inhalation air to be inhaled to the living body should be around 20° C. to about body temperature. Therefore, the control unit 22 may control the current value so that the temperature of the mixed gas delivered from the hydrogen gas delivery unit 8 becomes around 20° C. to about body temperature based on the temperature detected by the temperature sensor 10. A heating means may be separately provided so that the temperature of the mixed gas becomes around 20° C. to about body temperature without control of the current value by the control unit 22. A heating device may also be separately provided between the filter 12 and the living body.

### Second Embodiment

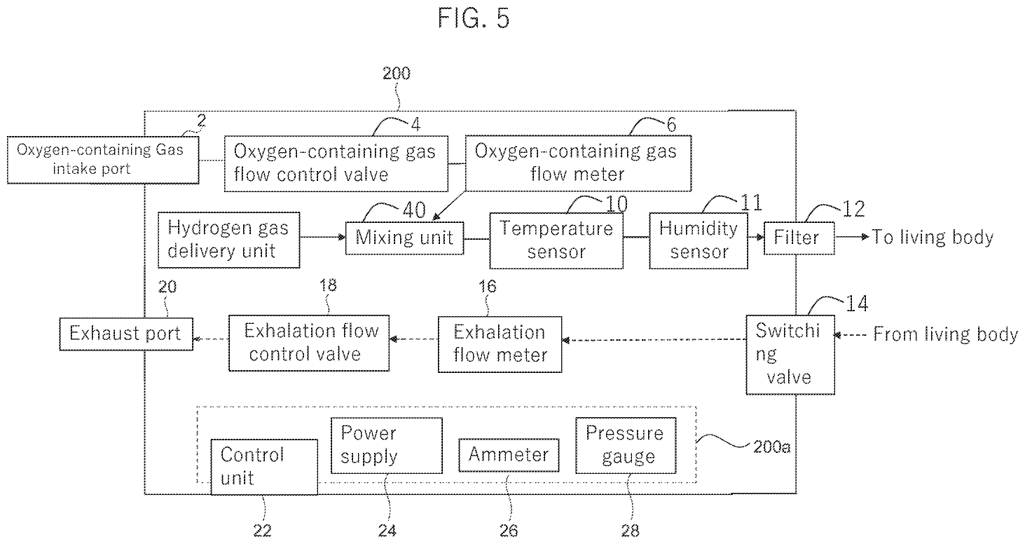

With reference to the drawings, a ventilator (an example of a gas supply apparatus) 200 of the second embodiment will be described in detail. As shown in FIG. 5, the ventilator 200 differs from the ventilator 100 of the first embodiment in that the hydrogen gas delivered from the hydrogen gas delivery unit 38 is mixed with the oxygen-containing gas in the mixing unit 40. That is, in this embodiment, the oxygen-containing gas and hydrogen gas are mixed outside the hydrogen gas delivery unit 38. In the ventilator 200, a humidity sensor 11 is provided between the temperature sensor 10 and the filter 12. Other components are similar to those of the ventilator 100 of the first embodiment and are therefore omitted from the description.

As shown in FIG. 5, the ventilator 200 has a mixing unit 40 between the hydrogen gas delivery unit 38 and the filter 12, where oxygen-containing gas and hydrogen gas are mixed. The oxygen-containing gas delivered from the oxygen-containing gas flow control valve 4 flows through the oxygen-containing gas flow meter 6 into the mixing unit 40.

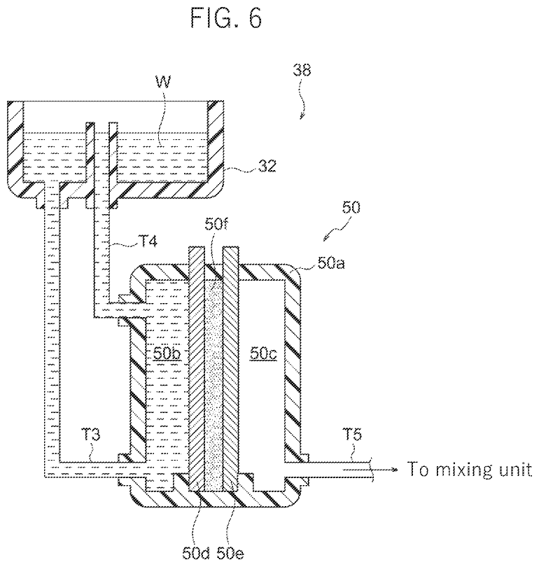

As shown in FIG. 6, the hydrogen gas delivery unit 38 of this embodiment is composed of the electrolyzer 50 and the water tank 32. The hydrogen gas delivery unit 38 is similar to the hydrogen gas delivery unit 8 of the first embodiment, except for the second chamber 50c of the electrolyzer 50. The second chamber 50c of the electrolyzer 50 has only an air delivery tube T5 as a tube connected to the outside. In this embodiment, the hydrogen gas taken into the second chamber 50c is delivered to the outside of the hydrogen gas delivery unit 38 through the air supply pipe T2 and flows into the mixing unit 40 without being mixed with other gases.

The hydrogen gas delivered from the hydrogen gas delivery unit 38 to the mixing unit 40 is mixed within the mixing unit 40 with the oxygen-containing gas that flows into the mixing unit 40 and is delivered to the filter 12 as mixed gas. The mixing unit 40 in this embodiment is not limited in terms of the configuration of the mixing unit 40, as long as a space is provided inside where the oxygen-containing gas and the hydrogen gas can be mixed.

The humidity sensor 11 detects the humidity of the mixed gas delivered from the mixing unit 40 to the filter 12, converts the detected humidity into an electrical signal, and transmits it to the control unit 22.

In the ventilator 200 of this embodiment, the control unit 22 adjusts the amount of hydrogen gas taken into the hydrogen gas delivery unit 38 by controlling the current value flowing to the pair of electrode plates 50d, 50e based on the flow rate of oxygen-containing gas, similarly to the first embodiment. Thus, the amount of hydrogen gas produced is increased or decreased in accordance with an increase or decrease in the flow rate of the oxygen-containing gas. As a result, the ventilator 200 can supply hydrogen gas of a highly accurate and stable hydrogen gas concentration to the body.

Since the humidity of the mixed gas is detected by the humidity sensor 11 in the ventilator 200 of this embodiment, the control unit 22 may control the current value flowing to the pair of electrode plates 50d, 50e based on the detected humidity. By controlling the current value, the production amount of hydrogen gas produced in the electrolyzer 50 can be controlled and the humidity of the mixed gas delivered from the hydrogen gas delivery unit 38 can be adjusted.

Here, the hydrogen gas produced by electrolysis is usually 100% hydrogen gas in terms of relative humidity (actual water vapor content/saturated water vapor content×100). In addition, the humidity of the inhalation air to be inhaled into the living body is usually 50% or more, preferably 100%, in terms of relative humidity. Therefore, the control unit 22 may control the current values of the pair of electrode plates 50d, 50e so that the relative humidity of the mixed gas delivered from the hydrogen gas delivery unit 38 is 50% or more.

In addition to adjusting the relative humidity of the mixed gas delivered from the hydrogen gas delivery unit 38 by controlling the current value by the control unit 22, for example, means may be provided to humidify the oxygen-containing gas (oxygen-containing gas mixed with hydrogen gas) delivered to the hydrogen gas delivery unit 38. If the absolute humidity of the inhaled air to be inhaled by the user of the ventilator 200 is insufficient even if the relative humidity of the mixed gas delivered from the hydrogen gas delivery unit 38 is high, a humidifier may be separately installed between the filter 12 and the living body. A configuration that can adjust the humidity of the mixed gas according to the relative humidity (or absolute humidity) of the inhaled air to be inhaled by the user may be provided as appropriate.

When controlling the current value based on humidity in this manner, the humidity of at least one of the gases, oxygen-containing gas, hydrogen gas, or mixed gas, may be detected by changing the position of the humidity sensor 11, and the current value may be controlled based on the detected humidity.

In the ventilator 200 of this embodiment, as in the ventilator 100 of the first embodiment, the oxygen-containing gas flow control valve 4 can be controlled based on the flow rate of the oxygen-containing gas, and the current value flowing to the electrode plates 50d, 50e can be controlled, and thereby the amount of hydrogen gas taken into the hydrogen gas delivery unit 38 can be adjusted. Therefore, the ventilator 200 can supply hydrogen gas of a highly accurate and stable hydrogen gas concentration to the body.

### Third Embodiment

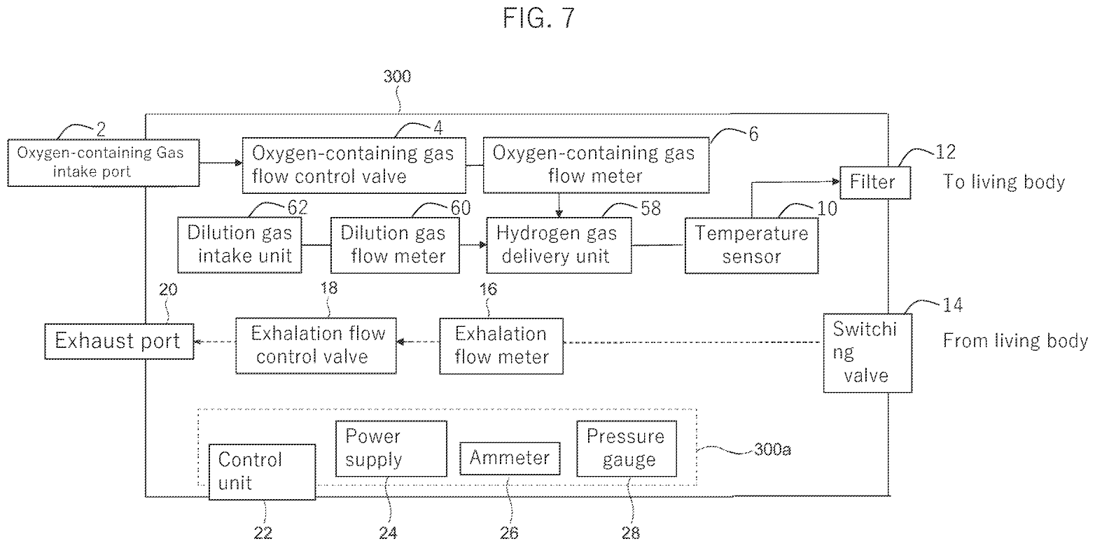

With reference to the drawings, ventilator 300 according to the third embodiment will be described in detail. As shown in FIG. 7, the ventilator 300 differs from the ventilator 100 of the first embodiment in that it has a dilution gas intake unit 62. In the ventilator 300, in addition to oxygen-containing gas, dilution gas delivered from the dilution gas intake unit 62 flows into the hydrogen gas delivery unit 58. In the ventilator 300, a dilution gas flow meter 60 is provided between the dilution gas intake unit 62 and the hydrogen gas delivery unit 58. Other components are similar to those of the ventilator 100 of the first embodiment and are therefore omitted from the description.

The dilution gas intake unit 62 is equipped with a dilution device (not shown), which consists of a cylinder or other container in which compressed oxygen-containing gas is sealed, and a dilution gas flow control valve (not shown). The oxygen-containing gas (dilution gas) sealed in the dilution device is a gas for diluting the hydrogen gas to be taken into the hydrogen gas delivery unit 58, apart from the oxygen-containing gas (oxygen-containing gas for inhalation) delivered from the oxygen-containing gas flow control valve 4. In the dilution gas intake unit 62, the dilution gas is taken from the dilution device and delivered outside the dilution gas intake unit 62 with the flow rate adjusted by the dilution gas flow control valve.

The dilution gas flow meter 60 detects the flow rate of dilution gas delivered from the dilution gas intake unit 62 to the hydrogen gas delivery unit 58, converts the detected flow rate into an electrical signal, and transmits it to the control unit 22.

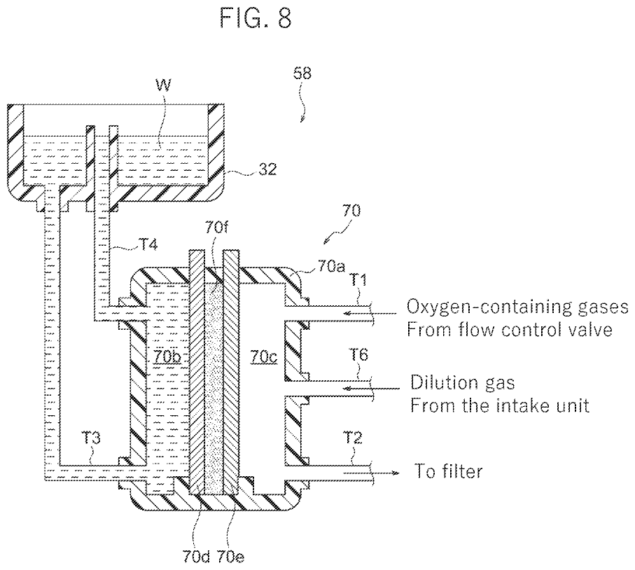

As shown in FIG. 8, the hydrogen gas delivery unit 58 of this embodiment is composed of the electrolyzer 70 and the water tank 32. The hydrogen gas delivery unit 58 is similar to the hydrogen gas delivery unit 8 of the first embodiment, except for the second chamber 70c of the electrolyzer 70. The second chamber 70c of the electrolyzer 70 is provided with a second intake pipe T6 through which the dilution gas flows in addition to the intake pipe T1 through which the oxygen-containing gas flows and the air supply pipe T2 through which the mixed gas is delivered. In this embodiment, the oxygen-containing gas and dilution gas that flow into the second chamber 70c are mixed in the second chamber 70c with respect to the hydrogen gas taken into the second chamber 70c, and are delivered to the filter 12 through the feed tube T2 as mixed gas. The mixed gas in this embodiment refers to a mixed gas of oxygen-containing gas, dilution gas, and hydrogen gas.

The control process of the current value executed by the control unit 22 of the ventilator 300 in this embodiment differs from the process shown in FIG. 4 in the following points. In the process of step S20, the control unit 22 detects the temperature of the mixed gas in which hydrogen gas, oxygen-containing gas, and dilution gas are mixed by an electrical signal transmitted from the temperature sensor 10.

If the flow rate of the dilution gas delivered from the dilution gas intake unit 62 is F_{D} (liters/s), the volume VD of the dilution gas flowing into the second chamber 70c of the electrolyzer 70 in 1 second (unit time) is equal to the flow rate F_{D}. Therefore, the volume per unit time of the mixed gas produced in the second chamber 70c in this system is V_{H}+Fₒ+F_{D}. Therefore, the following equation (10) is established.

Volume concentration of hydrogen gas (vol %)=(V_{H}/(V_{H}+Fₒ+F_{D}))×100 Equation (10)

In the ventilator 300 of this embodiment, the control unit 22 controls the dilution gas flow control valve to adjust the flow rate of the dilution gas based on the flow rate of the oxygen-containing gas so that the volume concentration of hydrogen gas calculated from the above equation (10) becomes the target production volume of hydrogen gas (e.g., greater than 0 vol % and within the range of 18.3 vol % or less). Furthermore, the control unit 22 controls the current values flowing to the electrode plates 70d and 70e of the electrolyzer 70 so that the volume concentration of hydrogen gas taken into the second chamber 70c of the electrolyzer 70 becomes the target production volume of hydrogen gas.

As described above, the control unit 22 of the ventilator 300 of this embodiment controls the oxygen-containing gas flow control valve 4 and the dilution gas flow control valve based on the flow rate of the oxygen-containing gas detected by the oxygen-containing gas flow meter so that the volume concentration of hydrogen gas in the mixed gas delivered from the hydrogen gas delivery unit 58 is within the predetermined range. The amount of hydrogen gas taken into the hydrogen gas delivery unit 58 is adjusted by controlling the current value flowing to the electrode plates 70d and 70e. Therefore, the ventilator 300 can supply hydrogen gas of a highly accurate and stable hydrogen gas concentration to the body.

In the ventilator 300 of this embodiment, the dilution gas intake unit 62 is equipped with a dilution device, but it is not limited to this configuration. For example, the dilution gas intake unit 62 may be configured to take in dilution gas from a cylinder or the like installed outside the ventilator 300 without a dilution device.

In the ventilator 300 of this embodiment, the configuration in which hydrogen gas, oxygen-containing gas, and dilution gas are mixed simultaneously in the second chamber 70c of the electrolyzer 70 is exemplified, but is not limited to this configuration. For example, the configuration may be that the hydrogen gas is mixed with the dilution gas after being mixed with the oxygen-containing gas, or the hydrogen gas is mixed with the oxygen-containing gas after being mixed with the dilution gas. Alternatively, the configuration may be such that the hydrogen gas is mixed only with the dilution gas and the oxygen-containing gas is delivered to the filter 12 without being mixed with the hydrogen gas.

### Fourth Embodiment

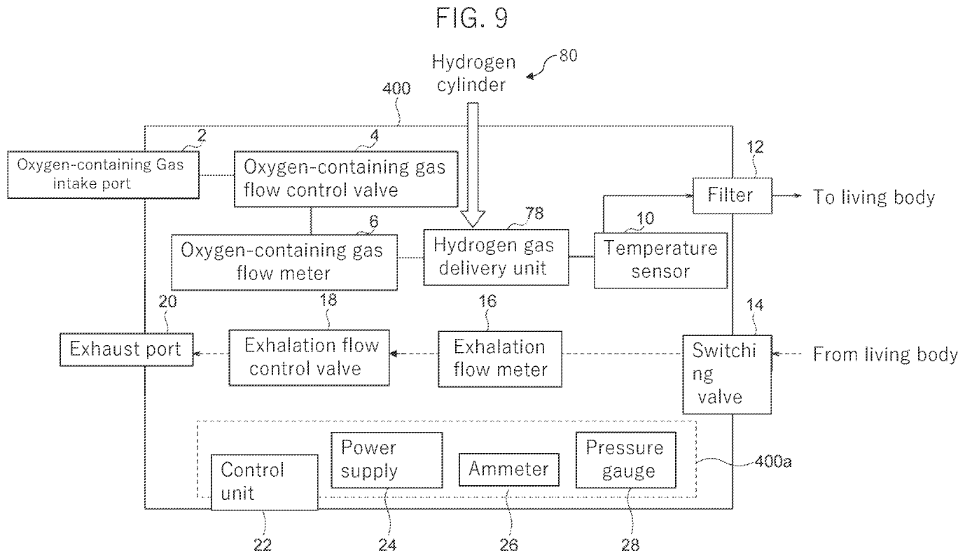

With reference to the drawings, a ventilator (an example of a gas supply apparatus) 400 according to the fourth embodiment will be described in detail. As shown in FIG. 9, the ventilator 400 differs from the ventilator 100 of the first embodiment in that the hydrogen gas delivery unit 78 is configured to take in hydrogen gas from a hydrogen cylinder 80 installed outside the ventilator 400. The other components are similar to those of the ventilator 100 of the first embodiment, so the description is omitted.

The hydrogen gas delivery unit 78 of this embodiment is not configured with an electrolyzer or a water tank, but has a space inside where, for example, oxygen-containing gas delivered from the oxygen-containing gas flow control valve 4 and hydrogen gas taken in from the hydrogen cylinder 80 can be mixed. The hydrogen gas delivery unit 78 is also provided with a mounting portion (not shown) for mounting the hydrogen cylinder 80, a hydrogen gas flow control valve (not shown) for controlling the flow rate of hydrogen gas taken in from the hydrogen cylinder 80, and a hydrogen gas flow meter (not shown) for detecting the flow rate of hydrogen gas flowing into the hydrogen gas delivery unit 78.

In this embodiment, the flow rate of hydrogen gas taken into the hydrogen gas delivery unit 78 from the hydrogen cylinder 80 is controlled by the hydrogen gas flow control valve. The hydrogen gas taken into the hydrogen gas delivery unit 78 from the hydrogen cylinder 80 is mixed in the hydrogen gas delivery unit 78 with the oxygen-containing gas that flows into the hydrogen gas delivery unit 78 and delivered to the filter 12 as mixed gas.

The control unit 22 of the ventilator 400 of this embodiment controls the hydrogen gas flow control valve based on the flow rate of the oxygen-containing gas so that the volume concentration of hydrogen gas in the mixed gas delivered from the hydrogen gas delivery unit 78 is within a predetermined range (e.g., greater than 0 vol % and 18.3 vol % or less), adjust the amount of hydrogen gas taken into the hydrogen gas delivery unit 78 from the hydrogen cylinder 80. Thus, the ventilator 400 can supply an appropriate amount of hydrogen gas to the body while maintaining safety.

### Fifth Embodiment

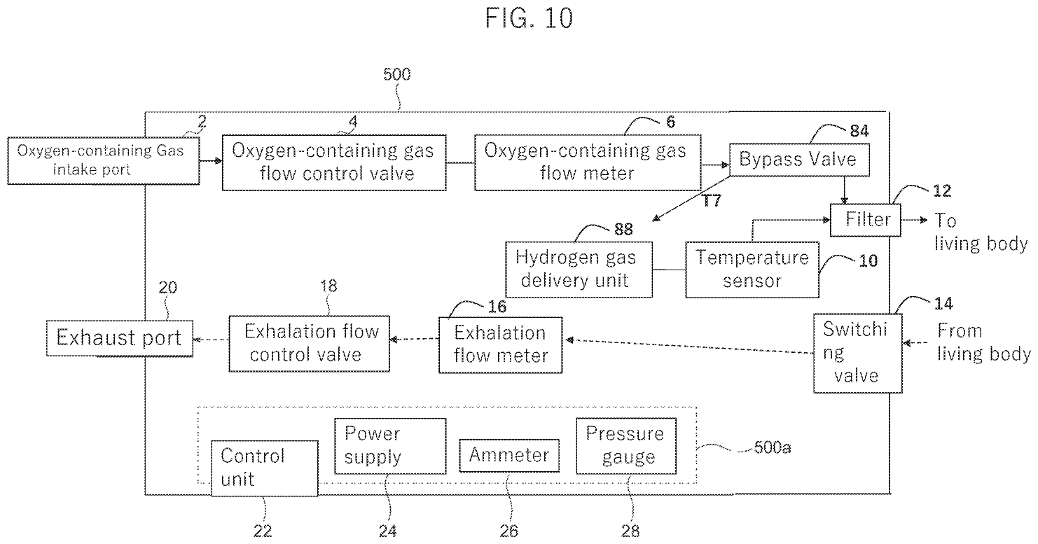

With reference to the drawings, a ventilator (an example of a gas supply apparatus) 500 according to the fifth embodiment will be described in detail. As shown in FIG. 10, the ventilator 500 differs from the ventilator 100 of the first embodiment in that a bypass valve (an example of a switching unit) 84 is provided downstream of the oxygen-containing gas flow control valve 4. The other components are similar to those of the ventilator 100 of the first embodiment, so the description is omitted.

As shown in FIG. 10, in the ventilator 500, oxygen-containing gas delivered from the oxygen-containing gas flow control valve 4 flows through the oxygen-containing gas flow meter 6 to the bypass valve 84. Downstream of the bypass valve 84, there is a first flow path T7 that delivers the oxygen-containing gas from the bypass valve 84 to the hydrogen gas delivery unit 88, and a second flow path T8 that delivers the oxygen-containing gas through the filter 12 without going through the hydrogen gas delivery unit 88 to the outside of the ventilator 500.

The bypass valve 84 is a valve that switches the direction of flow of the oxygen-containing gas between the first flow path T7 and the second flow path T8. The bypass valve 84 has a valve that opens and closes under the control of the control unit 22, for example, and the direction of flow of the oxygen-containing gas is controlled by the opening and closing of this valve. When the first flow path T7 is open, the oxygen-containing gas delivered from the bypass valve 84 flows into the hydrogen gas delivery unit 88, is mixed with hydrogen gas in the hydrogen gas delivery unit 88, and is delivered to the filter 12 as mixed gas. When the second flow path T8 is open, the oxygen-containing gas delivered from the bypass valve 84 is delivered to the filter 12 without being mixed with hydrogen gas.

In the ventilator 500, when the volume concentration of hydrogen gas delivered from the hydrogen gas delivery unit 88 exceeds a certain value, or when it is determined that it is harmful to supply said hydrogen gas to a living body (abnormal condition), the control unit 22 may control the bypass valve 84 to close the second flow path T8. This control allows the oxygen-containing gas delivered from the oxygen-containing gas flow control valve 4 to be delivered to the filter 12 without being delivered to the hydrogen gas delivery unit 88. This allows the ventilator 500 to function as a normal ventilator 500 in abnormal conditions.

As described above, the ventilator 500 of this embodiment can achieve the same effects as the ventilator 100 of the first embodiment and can also be made to respond to abnormal conditions such as harmful hydrogen gas being delivered.

In this embodiment, a configuration in which the bypass valve 84 has a valve that opens and closes under the control of the control unit 22 is exemplified, but the bypass valve 84 may also have a valve that opens and closes manually.

### Sixth Embodiment

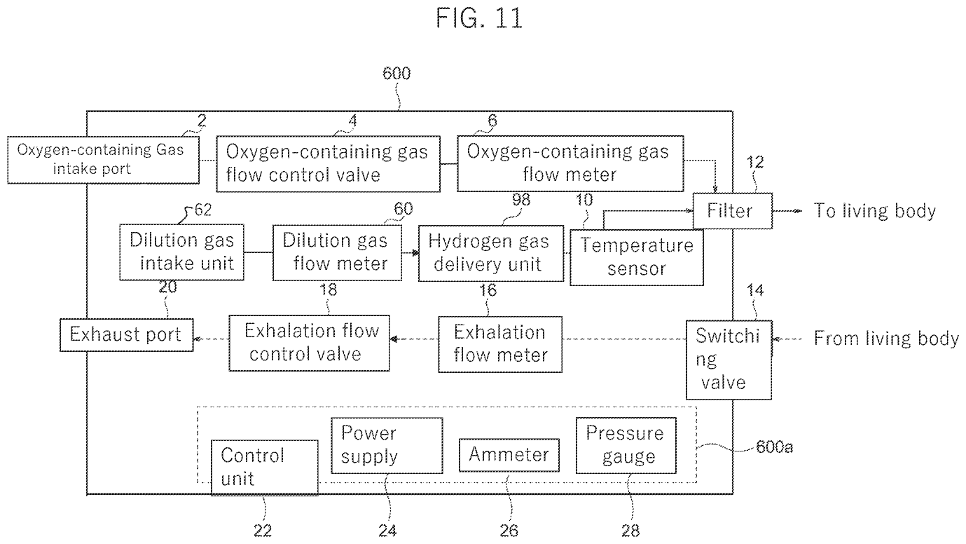

With reference to the drawings, the ventilator 600 according to the sixth embodiment will be described in detail. As shown in FIG. 11, the ventilator 600 has a dilution gas intake unit 62 as in the third embodiment. On the other hand, the ventilator 600 differs from the third embodiment in that only the dilution gas delivered from the dilution gas intake unit 62 flows into the hydrogen gas delivery unit 98, and the oxygen-containing gas is delivered to the filter 12 without going through the hydrogen gas delivery unit 98. The other components are similar to those of the ventilator 300 of the third embodiment, so the description is omitted.

The hydrogen gas delivery unit 98 of this embodiment differs from the hydrogen gas delivery unit 8 of the first embodiment in that dilution gas flows into the intake pipe T1 instead of oxygen-containing gas. In the hydrogen gas delivery unit 98 of this embodiment, the dilution gas that flows in from the intake pipe is mixed with the hydrogen gas taken into the hydrogen gas delivery unit 98 and delivered to the filter 12 through the delivery pipe as a mixed gas. Therefore, the mixed gas in this embodiment refers to the mixed gas of hydrogen gas and dilution gas.

The control process of the current value executed by the control unit 22 of the ventilator 600 in this embodiment differs in the following points of the process shown in FIG. 4. In the process of step S20, the control unit 22 detects the temperature of the mixed gas in which hydrogen gas and dilution gas are mixed by an electrical signal transmitted from the temperature sensor 10.

If the flow rate of the dilution gas delivered from the dilution gas intake unit 62 is F_{D} (liters/s), the volume VD of the dilution gas flowing into the hydrogen gas delivery unit 98 in one second (unit time) is equal to the flow rate F_{D}. Therefore, the volume per unit time of the mixed gas produced in the hydrogen gas delivery unit 98 in this embodiment is V_{H}+F_{D}. Therefore, the following equation (11) is established.

Volume concentration of hydrogen gas (vol %)=(V_{H}/(V_{H}+F_{D}))×100 Equation (11)

In the ventilator 600 of this embodiment, the control unit 22 controls the current value flowing through the electrode plates of the electrolyzer in the hydrogen gas delivery unit 98 based on the flow rate of the dilution gas so that the volume concentration of hydrogen gas calculated from the above equation (11) becomes the target production volume of hydrogen gas (e.g., greater than 0 vol % and within the range of 18.3 vol % or less).

As described above, the control unit 22 of the ventilator 600 of this embodiment controls the current value flowing to the electrode plates in the hydrogen gas delivery unit 98 based on the flow rate of the dilution gas detected by the dilution gas flow meter 60 so that the volume concentration of hydrogen gas in the mixed gas delivered from the hydrogen gas delivery unit 98 is within the predetermined range. By doing so, the amount of hydrogen gas taken into the hydrogen gas delivery unit 98 is adjusted. Therefore, the ventilator 600 can supply hydrogen gas of a highly accurate and stable hydrogen gas concentration to the body.

### OTHER EMBODIMENTS

In each of the above embodiments, a configuration in which the electrode plate provided in the first chamber is in contact with the diaphragm is exemplified, but for example, the electrode plate may be provided at a predetermined distance from the diaphragm. In the above embodiments, the configuration in which the electrode plate provided in the second chamber is in contact with the diaphragm is exemplified, but it is not necessary that they are crimped, but only that they are in contact to the extent that a water film is formed between the diaphragm and the electrode plate. The electrodes provided in the electrolyzer are not limited to a plate shape, but may be cylindrical, for example.

In each of the above embodiments, the configuration of the electrolyzer is not limited. In each of the above embodiments, an example is given of one diaphragm provided on the electrode plate, but the shape and number of diaphragms provided in the electrolyzer are not limited. The interior of the electrolyzer is not limited to being divided by the diaphragm, but may be divided by plastic or other materials.

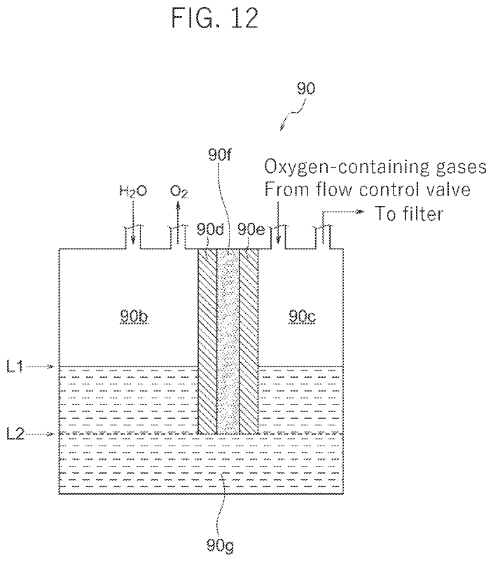

In each of the above embodiments, when the electrolyzed water is introduced into the cathode side of the electrolyzer, the case where the electrolyzed water is introduced into both the anode and cathode sides of the electrolyzer is also included. As an example of an electrolyzer in which the electrolyzed water is introduced to both the anode and cathode sides of the electrolyzer, an electrolyzer 90 as shown in FIG. 12 can be employed. The electrolyzer 90 shown in FIG. 12 has a housing, a first chamber 90b, a second chamber 90c, an electrode plate (anode) 90d in the first chamber 90b, an electrode plate (cathode) 90e in the second chamber 90c, and a diaphragm 90f separating both electrode plates 90d and 90e. A connecting portion 90g is provided in the lower part of the housing to connect the first chamber 90b and the second chamber 90c. Electrolyzed water flows into the first chamber 90b from a water tank, which is not shown in the figure, and is stored over both the first chamber 90b and the second chamber 90c through the connecting portion 90g to the first water level L1 (a position above the lower edge of the pair of electrode plates 90d, 90e and the septum 90f).

In this electrolyzer 90, electrolysis of the electrolyzed water is performed as follows. When an electric current flows through the electrode plate (anode) 90d, oxygen gas is generated in the first chamber 90b on the anode side and hydrogen gas is generated in the second chamber 90c on the cathode side. The oxygen gas and hydrogen gas generated at this time do not mix because the electrolyzed water stored up to the first water level L1 prevents them from coming and going between the first chamber 90b and the second chamber 90c. Therefore, the oxygen gas is delivered outside the first chamber without being mixed with the hydrogen gas, and the hydrogen gas is mixed with the oxygen-containing gas flowing into the second chamber without being mixed with the oxygen gas in the first chamber, and is delivered to the filter as mixed gas.

As the electrolysis is carried out for a long time, the water level of the electrolyzed water drops and reaches the second water level L2 to the bottom of the electrode plates 90d, 90e, the electrolyzed water becomes non-contact with the electrode plates 90d, 90e, and the generation of hydrogen gas and oxygen gas stops. At this time, because the connecting portion 90g is filled with the electrolyzed water up to the second water level L2, no gas flow occurs between the first chamber 90b and the second chamber 90c.

In each of the above embodiments, the configuration in which the control unit 22 causes the living body to inhale a predetermined amount of oxygen-containing gas within a predetermined time period in a single ventilation is exemplified, but for example, a configuration in which the control unit 22 causes the living body to inhale a variable amount of oxygen-containing gas at a predetermined pressure and within a predetermined time period in a single ventilation may be adopted. In this case, since the amount of oxygen-containing gas to be inhaled into the living body varies, the above-mentioned process of the control unit 22 calculating the current value should be repeatedly performed in one inhalation cycle.

In each of the above embodiments, a ventilator is illustrated as an example of a gas supply apparatus, but it is not limited to this. For example, a device that delivers gas over a space may be employed as a gas supply apparatus. In addition, it may be used not only for living organisms, but also for breathing organisms.

### REFERENCE SIGNS LIST

8, 38, 58, 78, 88, 98 Hydrogen gas delivery unit
10 Temperature sensor
11 Humidity sensor
22 Control unit
50, 70, 90 Electrolyzer
50d, 70d, 90d Electrode plate (electrode)
50e, 70e, 90e Electrode plate (electrode)
62 Dilution gas intake unit
84 Bypass valve (switching unit)
100, 200, 300, 400, 500, 600 Ventilator (gas supply apparatus)
T7 First flow path
T8 Second flow path
W Electrolyzed water

1. A gas supply apparatus that supplies oxygen-containing gas and hydrogen gas, comprising:
a hydrogen gas delivery unit that delivers said hydrogen gas taken in to outside of said gas supply apparatus; and
a control unit that adjusts an amount of said hydrogen gas taken into said hydrogen gas delivery unit, wherein
said control unit adjusts the amount of said hydrogen gas taken into said hydrogen gas delivery unit based on a flow rate of said oxygen-containing gas.

2. The gas supply apparatus according to claim 1, wherein
said hydrogen gas delivery unit generates and takes in said hydrogen gas by electrolysis of electrolyzed water performed using a pair of electrodes, and
said control unit adjusts the amount of said hydrogen gas taken into said hydrogen gas delivery unit by controlling a value of electric current flowing through said pair of electrodes based on the flow rate of said oxygen-containing gas.

3. The gas supply apparatus according to claim 2, wherein said control unit adjusts the amount of said hydrogen gas taken into said hydrogen gas delivery unit by controlling the value of said electric current flowing through said pair of electrodes based on the flow rate of said oxygen-containing gas delivered to said hydrogen gas delivery unit and mixed with said hydrogen gas.

4. The gas supply apparatus according to claim 1, further comprising a temperature sensor that detects a temperature of at least one of said oxygen-containing gas, said hydrogen gas, and a mixed gas of said oxygen-containing gas and said hydrogen gas,
wherein said control unit adjusts the amount of said hydrogen gas taken into said hydrogen gas delivery unit, based on the flow rate of said oxygen-containing gas and the temperature detected by said temperature sensor.

5. The gas supply apparatus according to claim 1, further comprising a humidity sensor that detects humidity of at least one of said oxygen-containing gas, said hydrogen gas, and a mixed gas of said oxygen-containing gas and said hydrogen gas,
wherein said control unit adjusts the amount of said hydrogen gas taken into said hydrogen gas delivery unit, based on the flow rate of said oxygen-containing gas and the humidity detected by said humidity sensor.

6. The gas supply apparatus according to claim 1, wherein said control unit adjusts the amount of said hydrogen gas taken into said hydrogen gas delivery unit so that a volume concentration of said hydrogen gas delivered from said hydrogen gas delivery unit becomes greater than 0 vol % and equal to or less than 18.3 vol %.

7. The gas supply apparatus according to claim 1, wherein said control unit adjusts the amount of said hydrogen gas taken into said hydrogen gas delivery unit so that a volume concentration of said hydrogen gas in a mixed gas in which said oxygen-containing gas and said hydrogen gas are mixed becomes a target concentration.

8. The gas supply apparatus according to claim 1, further comprising a first flow path that delivers said oxygen-containing gas to said hydrogen gas delivery unit, a second flow path that delivers said oxygen-containing gas to outside of said gas supply apparatus without causing said oxygen-containing gas to go through said hydrogen gas delivery unit, and a switching section that switches between said first flow path and said second flow path.

9. The gas supply apparatus according to claim 1, comprising a dilution gas intake unit that takes in dilution gas that is mixed with said hydrogen gas taken into said hydrogen gas delivery unit,
wherein said hydrogen gas delivery unit generates and takes in said hydrogen gas by electrolysis of electrolyzed water performed using a pair of electrodes, and
wherein said control unit adjusts the amount of said hydrogen gas taken into said hydrogen gas delivery unit by adjusting a flow rate of said dilution gas and controlling a value of electric current flowing through said pair of electrodes based on the flow rate of said oxygen-containing gas.

10. The gas supply apparatus according to claim 9, wherein said control unit adjusts the flow rate of said dilution gas taken into said dilution gas intake unit and the amount of said hydrogen gas taken into said hydrogen gas delivery unit so that a volume concentration of said hydrogen gas delivered from said hydrogen gas delivery unit becomes greater than 0 vol % and equal to or less than 18.3 vol %.

11. A gas supply apparatus that supplies oxygen-containing gas and hydrogen gas, comprising:
a hydrogen gas delivery unit that delivers said hydrogen gas taken in to outside of said gas supply apparatus;
a dilution gas intake unit that takes in dilution gas that is mixed with said hydrogen gas taken into said hydrogen gas delivery unit; and
a control unit that adjusts an amount of said hydrogen gas taken into said hydrogen gas delivery unit, wherein
said oxygen-containing gas is delivered to the outside of said gas supply apparatus without going through said hydrogen gas delivery unit,
said hydrogen gas delivery unit generates and takes in said hydrogen gas by electrolysis of electrolyzed water performed using a pair of electrodes, and
said control unit adjusts the amount of said hydrogen gas taken into said hydrogen gas delivery unit by controlling a value of electric current flowing through said pair of electrodes based on a flow rate of said dilution gas.

12. A gas supply method for supplying oxygen-containing gas and hydrogen gas, comprising:
a hydrogen gas generating process for generating said hydrogen gas by electrolysis of electrolyzed water; and
a mixing gas generating process for generating a mixed gas by mixing said hydrogen gas generated and taken in by said hydrogen gas generating process with said oxygen-containing gas, wherein
in said hydrogen gas generating process, an amount of said hydrogen gas to be generated is adjusted by controlling a value of electric current flowing in said electrolysis based on a flow rate of said oxygen-containing gas to be mixed with said hydrogen gas.

13. The gas supply method according to claim 12, wherein, in said hydrogen gas generating process, the amount of said hydrogen gas to be generated is adjusted by controlling the value of electric current flowing in said electrolysis so that a volume concentration of said hydrogen gas generated and delivered in said hydrogen gas generating process becomes greater than 0 vol % and equal to or less than 18.3 vol %.
